(19) United States
(12) Patent Application Publication
(10) Pub. No.: US 2014/0142466 A1
(43) Pub. Date: May 22, 2014

(54) PHYSICAL MOTION DETECTING DEVICE AND CONTROL METHOD FOR PHYSICAL MOTION DETECTING DEVICE

(71) Applicants: Yusuke Kawabe, Tokyo (JP); Kentaro Mori, Nagaokako-shi (JP)

(72) Inventors: Yusuke Kawabe, Tokyo (JP); Kentaro Mori, Nagaokako-shi (JP)

(73) Assignee: OMRON HEALTHCARE CO., LTD., Muko-shi, Kyoto (JP)

(21) Appl. No.: 14/129,872

(22) PCT Filed: Jun. 6, 2012
(86) PCT No.: PCT/JP2012/064507

§ 371 Date: Dec. 27, 2013

(30) Foreign Application Priority Data
Jul. 11, 2011 (JP) 2011-152717

(51) Int. Cl. A61B 5/11; A61B 5/00

(52) U.S. Cl. CPC A61B 5/112; A61B 5/1118; A61B 5/7278; A61B 5/7475; A61B 5/7221; A61B 5/742
USPC 600/595

(57) ABSTRACT

Whether one of a user's feet has touched the ground is detected on the basis of detection values from an acceleration sensor. In a single walking cycle, a period in which the user is standing on a first (the second) foot from a time when a detecting member detects that the first (second) foot has touched the ground to a time when the detecting member detects that a second (first) foot has touched the ground is defined as a first (second) stance period. A representative value for the detection values for each of the first and the second stance period is calculated on the basis of the detection values detected by the acceleration sensor. Whether or not a walking is an ascending (descending) walking is determined on the basis of a comparison result between the calculated representative values for the first and the second stance period.

### TECHNICAL FIELD

The present invention relates to a physical motion detecting device and a control method for the physical motion detecting device, and in particular, relates to a physical motion detecting device suitable for determining a walking is an ascending walking or a descending walking and a control method for the physical motion detecting device.

### BACKGROUND ART

Heretofore, there is a device which detects an ascending walking by using only an acceleration sensor. For example, there has been disclosed a pedometer which determines, on the basis of detection values from a three-axis acceleration sensor mounted at the waist of a user, that a walking is a stair ascending walking if an average of accelerations in the walking direction between two steps is greater than a predetermined value, and otherwise the walking is a stair descending walking (for example, see paragraph 0034 in Japanese Patent Laying-Open No. 2008-262522 (hereinafter, referred to as PTD 1)).

Further, there has been disclosed a human body-ascending/descending detecting device which projects a vector of x axis, which corresponds to the gravity axis, of a three-axis acceleration sensor onto a gravity axis which is identified at rest on the basis of detection values from the three-axis acceleration sensor mounted at the waist of a user, and determines that the human body is tilting forward, that is, ascending if an angle of the x axis relative to the gravity axis tilts to the positive direction to a certain degree or more, and determines that the human body is tilting backward, that is, descending if the angle tilts to the negative direction (for example, see paragraph 0025 and FIG. 2 in Japanese Patent Laying-Open No. 2008-173248 (hereinafter, referred to as PTD 2)).

Furthermore, there has been disclosed a stair ascending/descending detecting device which measures a walking pitch and an acceleration amplitude value in the gravity direction on the basis of detection values from a three-axis acceleration sensor mounted around the waist of a user, calculates an acceleration amplitude value from the measured walking pitch on the basis of data in a level ground walking characteristics table representing a relationship between the acceleration amplitude values and preliminarily stored walking pitches in a level ground walk, and determines that the walking is a stair descending walking if the measured acceleration amplitude value is greater than the calculated acceleration amplitude value, and otherwise the walking is stair ascending walking (for example, see paragraph 0026 and FIG. 3 in Japanese Patent Laying-Open No. 2008-154878 (hereinafter, referred to as PTD 3)).

### CITATION LIST

### Patent Document

### PTD 1: Japanese Patent Laying-Open No. 2008-262522

### PTD 2: Japanese Patent Laying-Open No. 2008-173248

### PTD 3: Japanese Patent Laying-Open No. 2008-154878

### SUMMARY OF INVENTION

### Technical Problem

According to the technique of PTD 1, it is necessary to calculate the acceleration in the walking direction from the three-axis acceleration. According to the technique of PTD 2, it is necessary to identify the gravity axis at rest, and it is necessary to calculate the acceleration of the identified gravity axis when walking. According to the technique of PTD 3, it is necessary to calculate the acceleration amplitude value in the gravity direction.

Thus, according to the techniques from PTD 1 to PTD 3, in an attempt to use physical motion detecting device such as a pedometer or an activity meter by mounting it freely to a user's body without fixing, it is necessary to firstly identify a predetermined direction, and thereby, the calculation amount becomes too excessive, which makes it difficult to determine a walking is an ascending walking or a descending walking in real time.

The present invention has been accomplished in view of the aforementioned problems, and one of objects of the present invention is to provide a physical motion detecting device capable of determining a walking is an ascending walking or a descending walking in real time, and a control method for the physical motion detecting device.

### Solution to Problem

To attain an object described above, according to an aspect of the present invention, there is provided a physical motion detecting device which includes a main body equipped with an accelerator sensor for detecting an acceleration of the main body and a control unit and is configured to detect physical motions of a user with the main body being mounted at a predetermined part of the user. The control unit includes a detecting member configured to detect whether or not one of the user's feet has touched the ground on the basis of detection values from the acceleration sensor. In a single walking cycle, a period in which the user is standing on a first foot from a time when the detecting member detects that the first foot has touched the ground to a time when the detecting member detects that a second foot has touched the ground is defined as a first stance period. In a single walking cycle, a period in which the user is standing on the second foot from a time when the detecting member detects that the second foot has touched the ground to a time when the detecting member detects that the first foot has touched the ground is defined as a second stance period. The control unit further includes a calculating member configured to calculate a representative value for the detection values detected by the acceleration sensor for each of the first stance period and the second stance period (for example, an integral for each period or an average of a maximum value and a minimum value for each period) on the basis of the detection values detected by the acceleration sensor, and a determining member configured to determine whether or not the walking is an ascending walking or a descending walking on the basis of a comparison result between the representative values calculated by the calculating member for the first stance period and the second stance period.

Preferably, the determining member determines whether or not the walking is an ascending walking or a descending walking on the basis of a comparison result whether or not a ratio of the representative value for the first stance period relative to the representative value for the second stance period is equal to or greater than a predetermined value.

More preferably, the physical motion detecting device further includes a notification unit configured to notify the user of a piece of predetermined information, and an input unit configured to receive an input of a piece of predetermined information from the user. The control unit further includes a notification controlling member configured to control the notification unit to notify the user of a result determined by the determining member, an input receiving member configured to receive from the input unit an input of a piece of true or false information representing a true or false indicator on the result notified by the notification member, and an adjusting member configured to adjust the predetermined value according to the true or false indicator represented by the piece of true or false information received by the input receiving member.

More preferably, the physical motion detecting device further includes an input unit configured to receive an input of a piece of predetermined information from the user. The control unit further includes an input receiving member configured to receive from the input unit an input for altering the predetermined value and an adjusting member configured to alter the predetermined value according to the input received by the input receiving member.

More preferably, the predetermined value is a value preliminarily determined according to a statistical approach. Preferably, the calculating member calculates an integral of the detection values for each of the first stance period and the second stance period as the representative value. Preferably, the calculating member calculates a value by using a maximum value and a minimum value of the detection values for each of the first stance period and the second stance period as the representative value.

According to another aspect of the present invention, there is provided a control method for controlling a physical motion detecting device which includes a main body equipped with an accelerator sensor for detecting an acceleration of the main body and a control unit and is configured to detect physical motions of a user with the main body being mounted at a predetermined part of the user. The control unit is configured to perform a step of detecting whether or not one of the user's feet has touched the ground on the basis of detection values from the acceleration sensor. In a single walking cycle, a period in which the user is standing on a first foot from a time when it is detected that the first foot has touched the ground to a time when it is detected that a second foot has touched the ground being defined as a first stance period. In a single walking cycle, a period in which the user is standing on the second foot from a time when it is detected that the second foot has touched the ground to a time when it is detected that the first foot has touched the ground being defined as a second stance period. The control unit is configured to further perform a step of calculating a representative value for the detection values detected by the acceleration sensor for each of the first stance period and the second stance period (for example, an integral for each period or an average of a maximum value and a minimum value for each period) on the basis of the detection values detected by the acceleration sensor, and a step of determining whether or not the walking is an ascending walking or a descending walking on the basis of a comparison result between the representative values calculated for the first stance period and the second stance period.

### Advantageous Effects of Invention

According to the physical motion detecting device and the control method for the physical motion detecting device of the present invention, whether or not one of the user's feet has touched the ground is detected on the basis of detection values from the acceleration sensor, a representative value for the detection values detected by the acceleration sensor for each of the first stance period and the second stance period is calculated on the basis of the detection values detected by the acceleration sensor, and whether or not the walking is an ascending walking or a descending walking is determined on the basis of a comparison result between the representative values calculated for the first stance period and the second stance period.

Thereby, whether or not a walking is an ascending walking or a descending walking can be determined on the basis of a comparison result between the representative values of the first stance period and the second stance period in one cycle. Since the ascending walking or the descending walking can be determined without the need of identifying the tilt of the physical motion detecting device, it is possible to reduce the calculation amount. As a result, it is possible to provide a physical motion detecting device capable of determining a walking is an ascending walking or a descending walking instantly during walking, and a control method for the physical motion detecting device.

### BRIEF DESCRIPTION OF DRAWINGS

FIG. 1 is a view illustrating an external appearance of an activity meter according to an embodiment of the present invention;

FIG. 2 is a view illustrating a situation where the activity meter according to the embodiment is in use;

FIG. 3 is a graph illustrating changes of a three-axis synthetic acceleration in walking;

FIG. 4 is a graph illustrating changes of a three-axis synthetic acceleration in an ascending walking;

FIG. 5 is a graph illustrating changes of a three-axis synthetic acceleration in a level ground walking;

FIG. 6 is an superimposed graph illustrating changes of the three-axis synthetic accelerations in the level ground walking, the ascending walking and a descending walking;

FIG. 7 is a table listing average ratios and standard deviations of integrals of acceleration during a left foot stance period and a right foot stance period in the level ground walking, the ascending walking and the descending walking;

FIG. 8 is a graph having average ratios of integrals of acceleration during the left foot stance period and the right foot stance period in the level ground walking, the ascending walking and the descending walking plotted for each subject;

FIG. 9 is a graph illustrating a range of average ratios of integrals of acceleration during the left foot stance period and the right foot stance period in the level ground walking and the ascending walking for each subject;

FIG. 10 is a block diagram illustrating a schematic configuration of the activity meter according to the embodiment;

FIG. 11 is a flowchart illustrating a procedure of a quantity of exercise calculating process executed by a control unit of the activity meter according to the embodiment; and

FIG. 12 is a flowchart illustrating a procedure of a quantity of exercise calculating process executed by the control unit of the activity meter according to a second embodiment.

### DESCRIPTION OF EMBODIMENTS

Hereinafter, embodiments of the present invention will be described in detail with reference to the drawings. It should be noted that the same or corresponding portions in the drawings will be given the same reference numerals and the description thereof will not be repeated.

In the embodiments of the present invention, a physical motion detecting device will be described as an activity meter capable of measuring not only steps but also a quantity of physical activity (also referred to as a quantity of exercise) for exercises and daily activities (for example, operating an electric vacuum cleaner, transporting a lightweight article, cooking and the like).

### First Embodiment

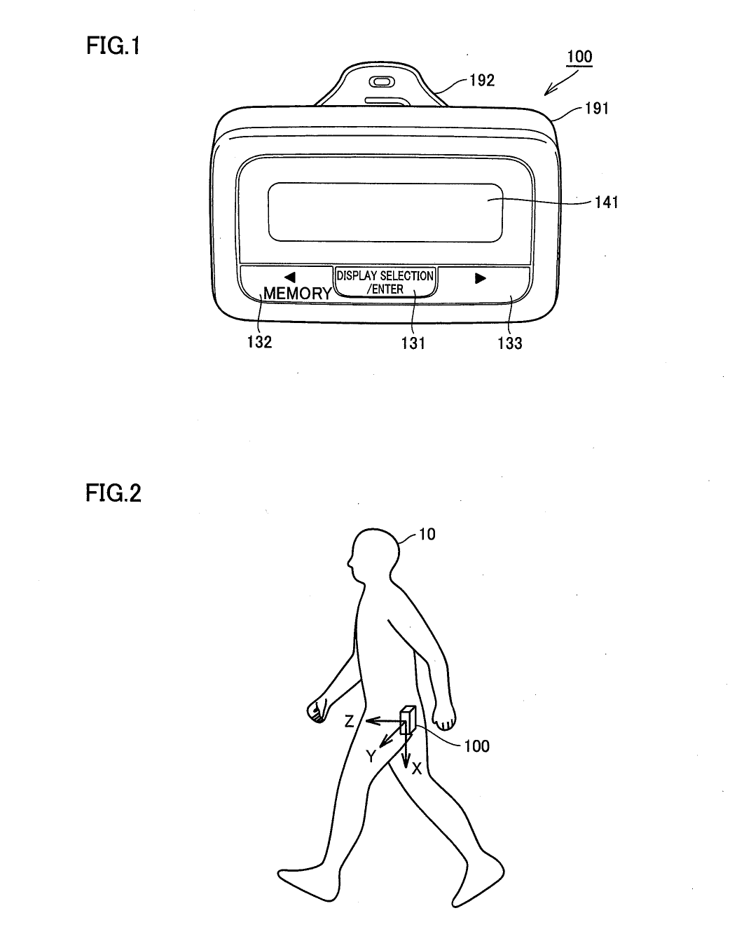

FIG. 1 is a view illustrating an external appearance of an activity meter 100 according to the embodiment of the present invention. With reference to FIG. 1, activity meter 100 mainly includes a main body 191 and a clip 192. Clip 192 is used to fix activity meter 100 on clothes or the like of a user.

Main body 191 is provided with a display selection/enter switch 131 which constitutes a part of an operation unit 130 to be described later, a left operation/memory switch 132, a right operation switch 133, and a display 141 which constitutes a part of a display unit 140 to be described later.

In the present embodiment, display 141 is described as a LCD display (Liquid Crystal Display) but not limited thereto, and it may be a display of other types, such as an EL (Electroluminescence) display and the like.

FIG. 2 is a view illustrating a situation where activity meter 100 according to the embodiment is in use. With reference to FIG. 2, activity meter 100 may be held by user 10 in a non-fixed manner by putting it in a pocket of trousers thereof, or may be mounted in a fixed manner, for example, at a belt around the waist of user 10 through clip 192.

Activity meter 100 may be designed as being held in a fixed manner or non-fixed manner at the other body parts of user 10, without being limited to the above.

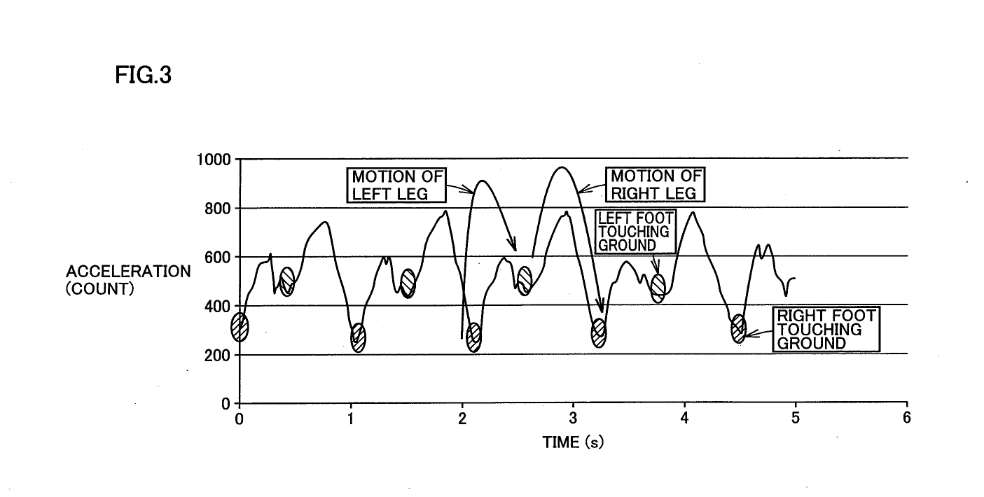

FIG. 3 is a graph illustrating changes of three-axis synthetic acceleration in walking. With reference to FIG. 3, the graph illustrates changes of three-axis synthetic acceleration output from an acceleration sensor 170 (to be described later) of activity meter 100 while the user is walking. For example, a time around 0 second, 1 second, 2.1 seconds, 3.2 seconds or 4.5 seconds in the time axis where the acceleration has a minimum value is the time when the right foot has touched the ground, and a time around 0.5 seconds, 1.5 seconds, 2.6 seconds or 3.8 seconds in the time axis is the time when the left foot has touched the ground.

Thus, the changes of acceleration in a right foot stance period from the time around 2.1 seconds where the right foot has touched the ground to the time around 2.6 seconds where the left foot has touched the ground are caused by the motions of the left foot, and the changes of acceleration in a left foot stance period from the time around 2.6 seconds where the left foot has touched the ground to the time around 3.2 seconds where the right foot has touched the ground are caused by the motions of the right foot.

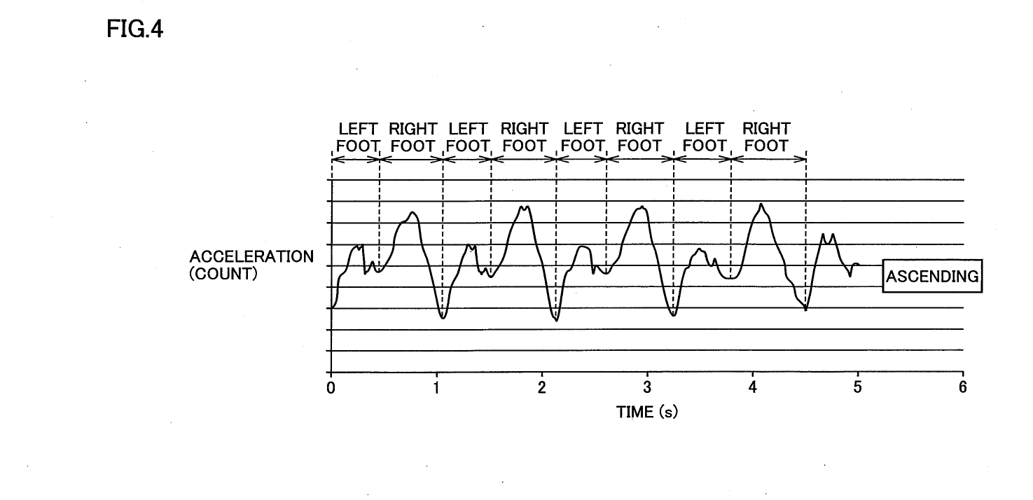

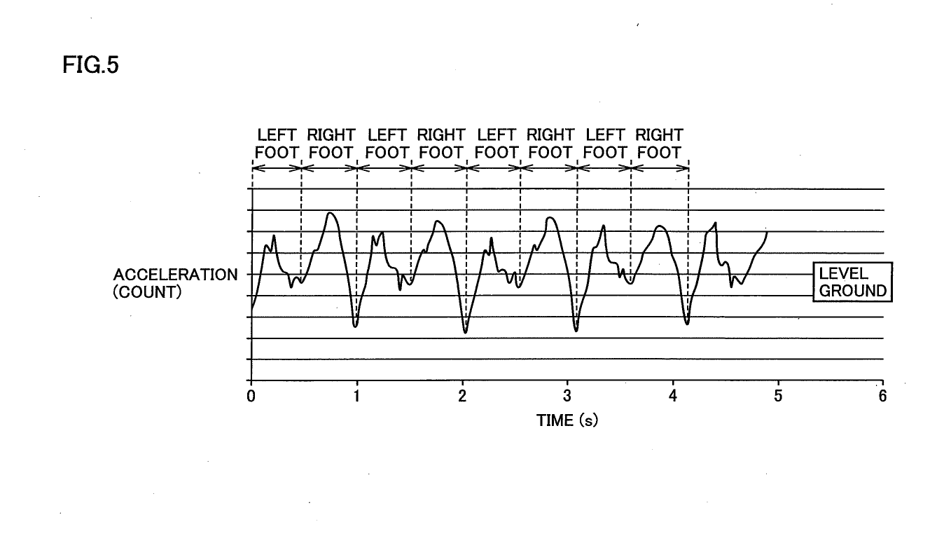

FIG. 4 is a graph illustrating changes of three-axis synthetic acceleration in an ascending walking, and FIG. 5 is a graph illustrating changes of three-axis synthetic acceleration in a level ground walking. With reference to FIGS. 4 and 5, it can be seen that in walking, the changes of acceleration while the right foot is moving are different from the changes of acceleration while the left foot is moving.

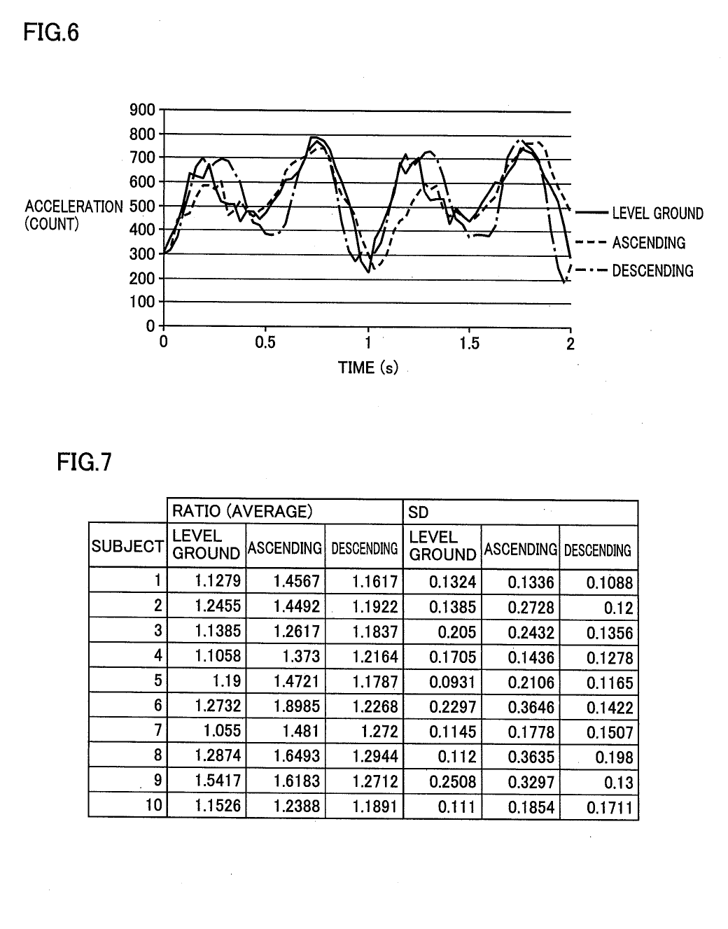

FIG. 6 is an superimposed graph illustrating changes of three-axis synthetic acceleration in the level ground walking, the ascending walking and a descending walking. With reference to FIG. 6, it can be seen that the acceleration difference between a first foot stance period and a second foot stance period in the ascending walking is greater than that between the first foot stance period and the second foot stance period in the level ground walking and the descending walking.

FIG. 7 is a table listing average ratios and standard deviations of integrals of acceleration during a left foot stance period and a right foot stance period in the level ground walking, the ascending walking and the descending walking. With reference to FIG. 7, it can be seen that for 8 subjects among 10 subjects except for subjects No. 3 and No. 10, the average ratio of integrals of acceleration during the left foot stance period and the right foot stance period in the ascending walking is obviously greater than the average ratio in the level ground walking and the descending walking. Thus, it is possible to determine whether a walking is an ascending walking or a level ground or a descending walking on the basis of a ratio of an acceleration of the left foot and an acceleration of the right foot.

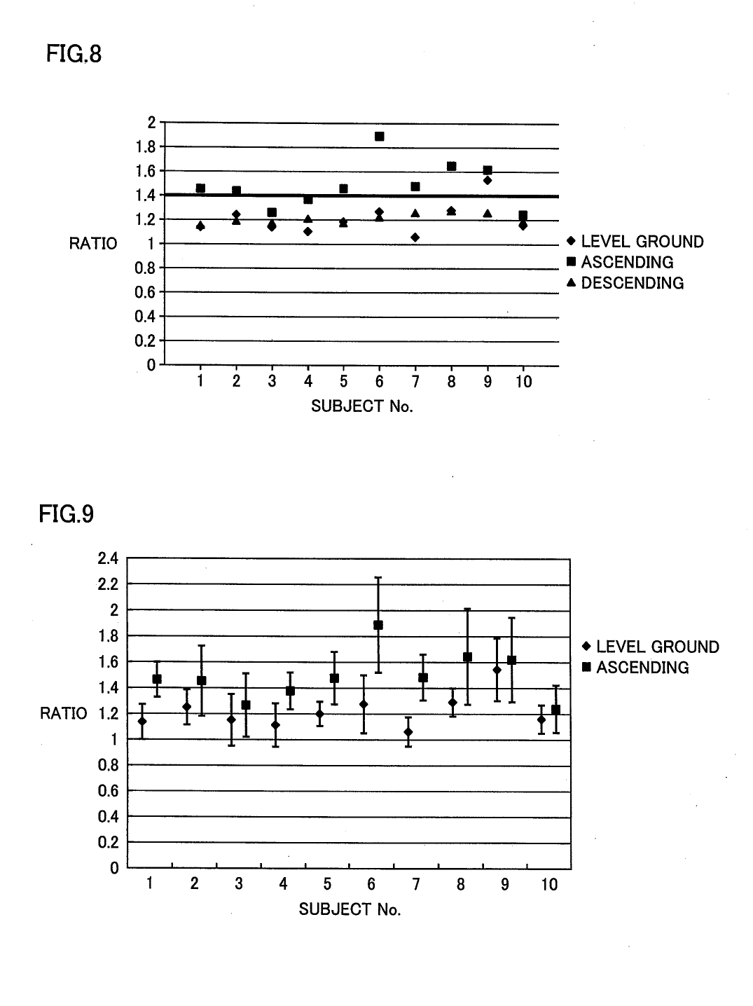

FIG. 8 is a graph having average ratios of integrals of acceleration during the left foot stance period and the right foot stance period in the level ground walking, the ascending walking and the descending walking plotted for each subject. FIG. 9 is a graph illustrating a range of average ratios of integrals of acceleration during the left foot stance period and the right foot stance period in the level ground walking and the ascending walking for each subject.

With reference to FIGS. 8 and 9, if a threshold value is set to 1.4, it is substantially possible to distinguish the ascending walking and the level ground walking. It can be seen that most ratios for the ascending walking are equal to or greater than 1.4 and most ratios for the level ground walking are smaller than 1.4.

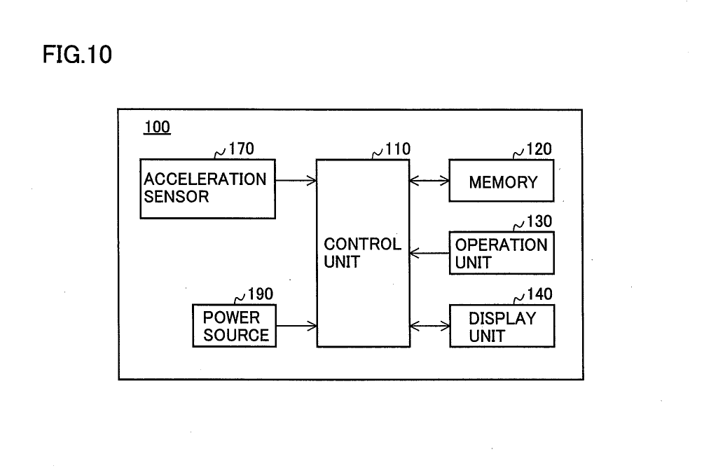

FIG. 10 is a block diagram illustrating a schematic configuration of activity meter 100 according to the present embodiment. With reference to FIG. 10, activity meter 100 includes a control unit 110, a memory 120, operation unit 130, display unit 140, an acceleration sensor 170, and a power source 190. Activity meter 100 may also include an audio unit for outputting sounds and an interface for communicating with an external computer.

Control unit 110, memory 120, operation unit 130, display unit 140, acceleration sensor 170, and power source 190 are disposed inside main body 191 described with reference to FIG. 1.

Operation unit 130 includes display selection/enter switch 131, left operation/memory switch 132 and right operation switch 133 which are described with reference to FIG. 1, and is configured to transmit operation signals representing operations performed on these switches to control unit 110.

A semiconductor sensor using MEMS (Micro Electro Mechanical Systems) technique is used as acceleration sensor 170 but it is not limited thereto, and it may be a sensor of another type, such as a mechanical or optical one. In the present embodiment, acceleration sensor 170 is configured to output to control unit 110 detection signals representing accelerations in three axes, respectively. However, acceleration sensor 170 is not limited to a three-axis acceleration sensor, and it may be a uniaxial acceleration sensor or a biaxial acceleration sensor.

Memory 120 includes a non-volatile memory such as a ROM (Read Only Memory) (for example, a flash memory), and a volatile memory such as a RAM (Random Access Memory) (for example, SDRAM (synchronous Dynamic Random Access Memory)).

Memory 120 is configured to store data of a program for controlling activity meter 100, data used to control activity meter 100, setting data for setting various functions of activity meter 100, and data of measurement results of the number of steps, the quantity of physical activity and the like every predetermined time (for example, every day). Memory 120 is also used as a working memory or the like at the time when the program is being executed.

Control unit 110 includes a CPU (Central Processing Unit), and is configured to follow the program stored in memory 120 for controlling activity meter 100 to control memory 120 and display unit 140 on the basis of detection signals from acceleration sensor 170 in response to operation signals from operation unit 130.

Display unit 140 includes display 141 described with reference to FIG. 1, and is configured to control display 141 to display a piece of predetermined information in accordance with a control signal from control unit 110.

Power source 190 includes a replaceable battery, and is configured to supply power from the battery to each component such as control unit 110 in activity meter 100 which needs electric power to operate.

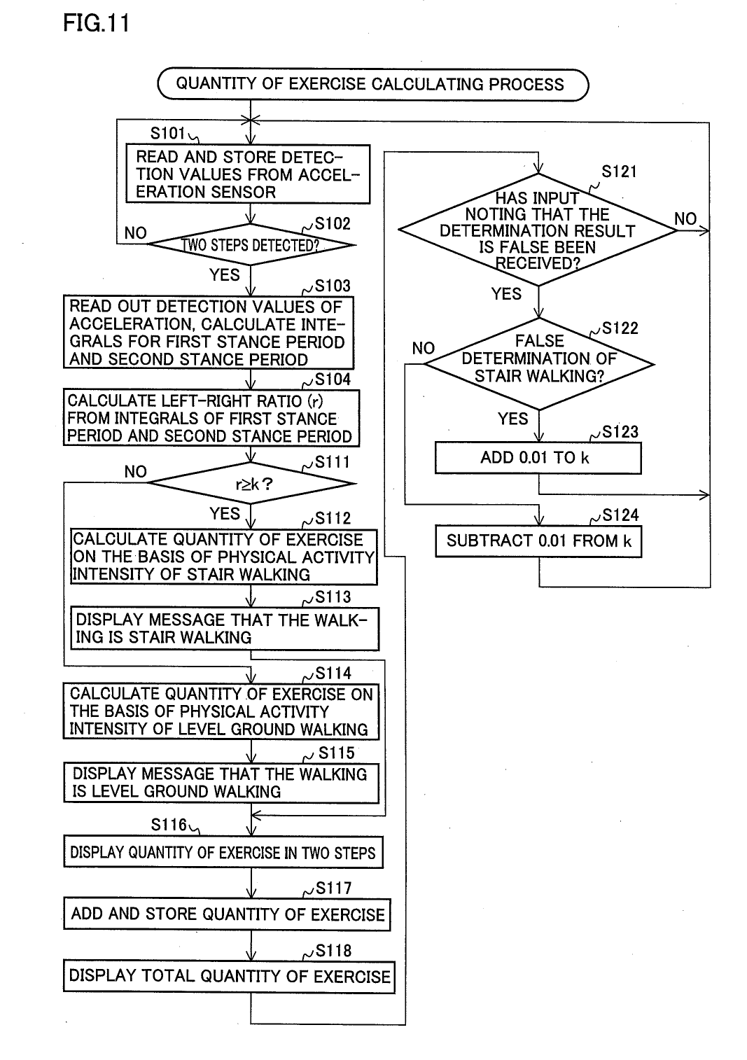

FIG. 11 is a flowchart illustrating a procedure of a quantity of exercise calculating process executed by control unit 110 of activity meter 100 according to the present embodiment. With reference to FIG. 11, at step S101, control unit 110 reads detection values represented by detection signals from acceleration sensor 170 for each sampling cycle and causes memory 120 to store the detection values.

Next, at step S102, control unit 110 determines whether or not a two-step walking has been detected. If a two-step walking has not been detected (NO at step S102), control unit 110 repeats the processing of step S101.

On the other hand, if a two-step walking has been detected (YES at step S102), then at step S103, control unit 110 reads out the detection values of acceleration stored in memory 120, and calculates a pseudo-integral da for a first stance period where one foot is standing in a first step and a pseudo-integral db for a second stance period where the other foot is standing in a second step. The pseudo-integrals may be calculated for each period, for example, by adding detection values for each sampling cycle in each period.

Thereafter, at step S104, control unit 110 calculates a left-right ratio r from the pseudo-integrals of the first and second stance periods.

Specifically, at the time when a predetermined number of steps (for example 10 steps) has been detected, control unit 110 first compares which integral of the pseudo-integral da for the first stance period and the pseudo-integral db for the second stance period is greater, determines which foot has greater integrals in its stance period, and sets the pseudo-integral of acceleration of the foot which has greater integrals in the stance period as dl and the pseudo-integral of acceleration of the other foot which has smaller integrals in the stance period as ds so as to calculate the left-right ratio r=dl/ds.

It is acceptable that control unit 110 sets the greater integral of the pseudo-integrals da and db as dl and the smaller one as ds so as to calculate the left-right ratio r=dl/ds for each two-step walking. Generally, r is calculated for each subject in such a way that the pseudo-integral in the stance period of a foot having a greater pseudo-integral of acceleration is set as numerator and the pseudo-integral in the stance period of the other foot having a smaller pseudo-integral of acceleration is set as denominator.

Then, at step S111, control unit 110 determines whether or not the left-right ratio r is equal to or greater than k. In the present embodiment, as mentioned in FIG. 8, the initial value of k is set to 1.4.

If it is determined that r is equal to or greater than k (YES at step S111), the walking is determined to be an ascending walking, and at step S112, control unit 110 calculates the quantity of exercise on the basis of a physical activity intensity of stair ascending walking.

Specifically, the physical activity intensities of a stair ascending walking, a level ground walking and a stair descending walking, for example, are set to 8.0 METs, 3.0 METs and 3.0 METs, respectively on the basis of descriptions in a reference document (“Exercise and Physical Activity Reference for Health Promotion 2006” (July, 2006) by Preparation Committee for Recommended Exercise Allowance and Exercise Guidelines).

Using the physical activity intensity mentioned above, the quantity of exercise EV (exercise (Ex) for every predetermined cycle (for example, every two steps) is calculated by the equation: EV (Ex)=Σ(Es×ET), where Es (METs) denotes the physical activity intensity, and ET (hour) denotes the lasting time of each physical motion.

The time for the two-step walking is calculated as ET (hour), and since Es=8.0 (METs), the quantity of exercise is calculated as EV (Ex)=8.0 (METs)×ET (hour).

Thereafter, at step S113, control unit 110 displays on display unit 140 a message that the walking is a stair walking. It is acceptable to display a message that the walking is an ascending walking. Thereafter, control unit 110 proceeds to execute the processing of step S116.

On the other hand, if it is determined that r is less than k (NO at step S111), then the walking is determined as a walking (level ground walking or descending walking) other than the ascending walking, and at step S114, control unit 110 calculates the quantity of exercise on the basis of the physical activity intensity of the level ground walking.

Here, the time for the two-step walking is calculated as ET (hour), and since Es=3.0 (METs), the quantity of exercise is calculated as EV (Ex)=3.0 (METs)×ET (hour).

Thereafter, at step S115, control unit 110 displays on display unit 140 a message that the walking is a level ground walking. It is acceptable to display the message that the walking is not a stair ascending walking. Thereafter, control unit 110 proceeds to execute the processing of step S116.

At step S116, control unit 110 displays on display unit 140 the quantity of exercise of the two-step walking calculated at step S112 or step S114. At step S117, control unit 110 adds all the quantity of exercise and stores the total quantity of exercise in memory 120, and at step S118, control unit 110 displays the total quantity of exercise on display unit 140.

Next, at step S121, control unit 110 determines whether or not an input denoting that the determination result displayed at step S113 or step S115 is false has been received from operation unit 113. If it is determined that such input has not been received (NO at step S121), control unit 110 returns back to execute the processing of step S101.

On the other hand, if it is determined that an input denoting that the determination result is false has been received (YES at step S121), then at step S122, control unit 110 determines whether or not the input denoting that the determination result is false is made against the determination result denoting that the walking is a stair walking.

If it is determined that the input denoting that the determination result is false is made against the determination result denoting that the walking is a stair walking (YES at step S122), then at step S123, control unit 110 adds 0.01 to a threshold value k of the left-right ratio.

On the other hand, if it is determined that the input denoting that the determination result is false is not made against the determination result denoting that the walking is a stair walking, in other words, if it is determined that the input denoting that the determination result is false is made against the determination result denoting that the walking is a level ground walking (NO at step S122), then at step S124, control unit 110 subtracts 0.01 from the threshold value k of the left-right ratio. After step S123 or step S124, control unit 110 returns back to execute the processing of step S101.

### Summary of First Embodiment

(1) As mentioned above, activity meter 100 in the first embodiment is such a device that includes main body 191 equipped with acceleration sensor 170 for detecting an acceleration of main body 191 and control unit 110, and is configured to detect physical motions of user 10 with main body 191 being mounted at a predetermined part of the user. As mentioned at step S102, whether or not a foot of the user has touched the ground is detected by control 110 on the basis of the detection values from acceleration sensor 170.

The first stance period refers to a period in a single walking cycle in which the user is standing on the first foot from a time when it is detected that the first foot has touched the ground to a time when it is detected that the second foot has touched the ground. The second stance period refers to a period in a single walking cycle in which the user is standing on the second foot from a time when it is detected that the second foot has touched the ground to a time when it is detected that the first foot has touched the ground.

As mentioned at step S103, control unit 110 calculates the pseudo-integral of the detection values by acceleration sensor 170 in each of the first stance period and the second stance period on the basis of the detection values by acceleration sensor 170, and as mentioned at step S104 and step S111, determines whether or not the walking is an ascending walking on the basis of the ratio between the calculated pseudo-integral of the first stance period and the calculated pseudo-integral of the second stance period.

Thereby, whether or not the walking is an ascending walking can be determined according to the calculated ratio between the pseudo-integral of the first stance period and the pseudo-integral of the second stance period in one cycle. Since the ascending walking or the descending walking can be determined without the need to identify the tilt of the physical motion detecting device, it is possible to reduce the calculation amount. As a result, it is possible to determine that a walking is an ascending walking instantly during walking.

(2) As mentioned at step S104 and step S111, control unit 110 determines whether or not the walking is an ascending walking on the basis of a comparison result whether or not the ratio between the pseudo-integral of the first stance period and the pseudo-integral of the second stance period is equal to or greater than a predetermined value (for example, 1.4).

(3) Activity meter 100 further includes display unit 140 configured to notify the user of a piece of predetermined information, and operation unit 130 configured to receive an input of a piece of predetermined information from the user. As mentioned at step S113 and step S115, control unit 110 controls display unit 140 to notify the determination result whether or not the walking is an ascending walking, and as mentioned at step S121 and step S122, the input of a piece of true-false information denoting whether the notified result is true or false is received from operation unit 130, and as mentioned at step S123 and step S124, the predetermined value is adjusted in accordance with the received piece of true-false information.

Thereby, it is possible to make the determination of the ascending walking more precisely in accordance with the characteristics such as the walking habit and the like of the user.

(4) Furthermore, the predetermined value is a value preliminarily determined according to a statistical approach.

(5) As mentioned at step S103, control unit 110 calculates the pseudo-integral of the detection values in each of the first stance period and the second stance period as a representative value.

### Second Embodiment

In the first embodiment, the left-right ratio r of a two-step walking is calculated for every two steps so as to determine whether or not a walking is an ascending walking. In the second embodiment, a left-right ratio r relative to a previous step is calculated for every one step so as to determine whether or not a walking is an ascending walking.

In the first embodiment, the detection values by acceleration sensor 170 for each of the first stance period and the second stance period in each sampling cycle are added so as to calculate the pseudo-integral. In the second embodiment, the pseudo-integral is calculated from a maximum acceleration detection value and a minimum acceleration detection value by acceleration sensor 170 for each of the first stance period and the second stance period.

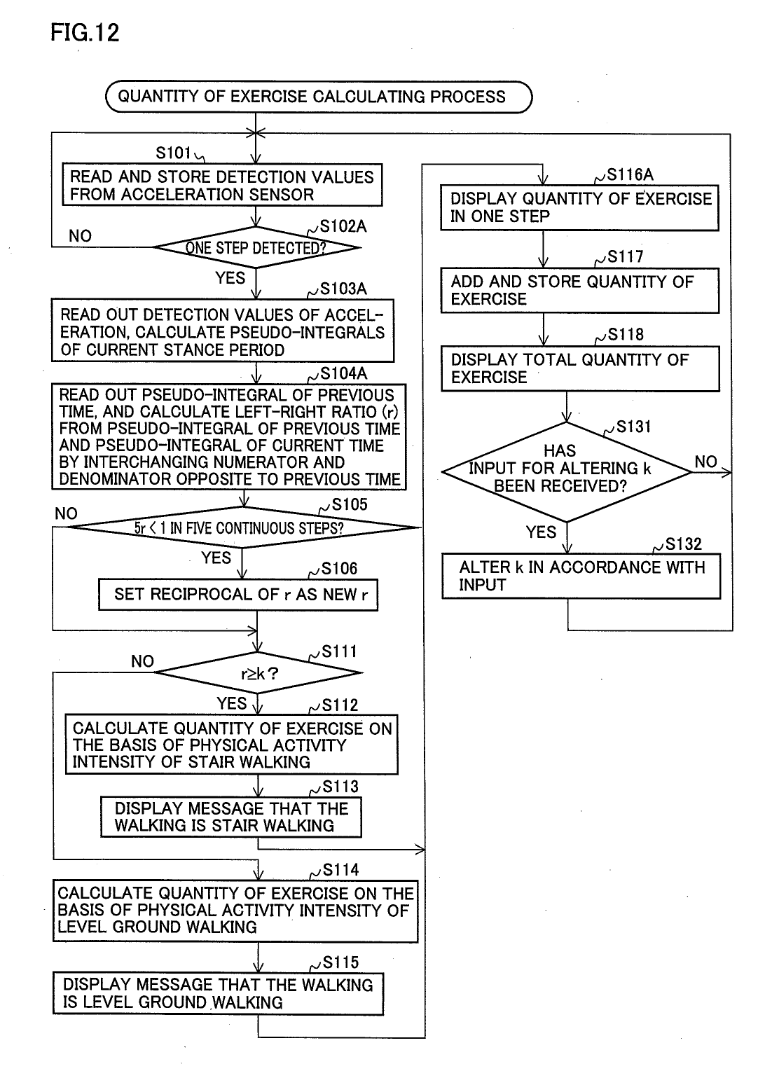

FIG. 12 is a flowchart illustrating a procedure of a quantity of exercise calculating process executed by control unit 110 of activity meter 100 according to the second embodiment. With reference to FIG. 12, step S101 is the same as that in FIG. 11.

Next, at step S102A, control unit 110 determines whether or not a one-step walking has been detected. If the one-step walking has not been detected (NO at step S102A), control unit 110 repeats the processing of step S101.

On the other hand, if the one-step walking has been detected (YES at step S102A), then at step S103A, control unit 110 reads out the detection values of acceleration stored in memory 120, and calculates a pseudo-integral df of current stance period. The pseudo-integral df is calculated by df=(|amax|+|amin|)×T, where T is a time duration of the one-step walking, amax is the maximum acceleration detection value of acceleration in the time duration, and amin is the minimum acceleration detection value of acceleration in the time duration.

In the above description, it is assumed both the maximum acceleration detection value amax and the minimum acceleration detection value amin are never less than zero. In the case where the maximum acceleration detection value amax and the minimum acceleration detection value amin may be less than zero, the pseudo-integral df is calculated by df=|amax+amin|×T.

Thereafter, at step S104A, control unit 110 reads the pseudo-integral dg of a previous time, and calculates the left-right ratio r from the pseudo-integral dg of the previous time and the pseudo-integral df of the current time by interchanging numerator and denominator opposite to those in the previous time. Specifically, if r is calculated in the previous time by r=df/dg, then r is calculated in the current time by r=dg/df; if r is calculated in the previous time by r=dg/df, then r is calculated in the current time by r=df/dg. Accordingly, r is calculated for each subject in such a way that the pseudo-integral in the stance period of a foot having a greater pseudo-integral of acceleration is used as numerator and the pseudo-integral in the stance period of the other foot having a smaller pseudo-integral of acceleration is used as denominator.

Thereafter, at step S105, whether or not r is smaller than 1 in a predetermined number of continuous steps (for example, 5 steps), in other words, whether or not there is a possibility that the left foot and the right foot are reversed in calculating the left-right ratio r is determined.

If it is determined that r is smaller than 1 in the predetermined number of continuous steps (YES at step S105), then at step S106, control unit 110 sets the reciprocal of r as a new r.

If it is determined that r is not smaller than 1 in the predetermined number of continuous steps (NO at step S105), and as well as after step S106, the control unit. 110 executes the same processing as that from step S111 to step S115 in FIG. 11.

At step S116A subsequent to step S113 or step S115, control unit 110 displays on display unit 140 the quantity of exercise of the one-step walking calculated at step S112 or step S114. Thereafter, control unit 110 executes the same processing as that in step S117 and step S118 in FIG. 11.

Thereafter, at step S131, control unit 110 determines whether or not an input for altering the threshold value k of the left-right ratio has been received from operation unit 130. If it is determined that such input has not been received (NO at step S131), control unit 110 returns back to execute the processing of step S101.

On the other hand, if it is determined that the input for altering k has been received (YES at step S131), then at step S132, control unit 110 alters the threshold value k of the left-right ratio in accordance with the input. Thereafter, control unit 110 returns back to execute the processing of step S101.

### Summary of Second Embodiment

As mentioned above, in addition to the effects provided by activity meter 100 described in the first embodiment, activity meter 100 according to the second embodiment provides additional effects as follows.

(1) Activity meter 100 according to the second embodiment is such a device that includes main body 191 equipped with acceleration sensor 170 for detecting an acceleration of main body 191 and control unit 110, and is configured to detect physical motions of user 10 with main body 191 being mounted at a predetermined part of the user. As mentioned at step S102A, whether or not a foot of the user has touched the ground is detected by control 110 on the basis of the detection values from acceleration sensor 170.

The first stance period refers to a period in a single walking cycle in which the user is standing on the first foot from a time when it is detected that the first foot has touched the ground to a time when it is detected that the second foot has touched the ground. The second stance period refers to a period in a single walking cycle in which the user is standing on the second foot from a time when it is detected that the second foot has touched the ground to a time when it is detected that the first foot has touched the ground.

As mentioned at step S103A, control unit 110 calculates the pseudo-integral of the detection values by acceleration sensor 170 in each of the first stance period and the second stance period on the basis of the detection values by acceleration sensor 170, and as mentioned at step S104A and step S111, determines whether or not the walking is an ascending walking on the basis of the ratio between the calculated pseudo-integral of the first stance period and the calculated pseudo-integral of the second stance period.

Thereby, whether or not the walking is an ascending walking can be determined according to the calculated ratio between the pseudo-integral of the first stance period and the pseudo-integral of the second stance period in one cycle. Since the ascending walking can be determined without the need to identify the tilt of the physical motion detecting device, it is possible to reduce the calculation amount. As a result, it is possible to determine that a walking is an ascending walking instantly during walking.

(2) As mentioned at step S104A and step S111, control unit 110 determines whether or not the walking is an ascending walking on the basis of a comparison result whether or not the ratio between the pseudo-integral of the first stance period and the pseudo-integral of the second stance period is equal to or greater than a predetermined value (for example, 1.4).

(3) Activity meter 100 further includes operation unit 130 configured to receive an input of a piece of predetermined information from the user. As mentioned at step S131, if an input for altering the predetermined value is received from operation unit 130, then as mentioned at step S132, control unit 110 alters the predetermined value in accordance with the received input.

Thereby, it is possible to make the determination of the ascending walking more precisely in accordance with the characteristics such as the walking habit and the like of the user.

(4) Furthermore, the predetermined value is a value preliminarily determined according to a statistical approach.

(5) Control unit 110 calculates the pseudo-integral of the detection values in each of the first stance period and the second stance period as the representative value.

(6) Control unit 100 calculates the pseudo-integral by using the maximum value and the minimum value of the detection values in each of the first stance period and the second stance period as the representative value.

(7) In comparison to the first embodiment, the calculation amount for obtaining the pseudo-integrals is less in the second embodiment, and thereby, it is possible to save the power of activity meter 100.

[Modification]

(1) Activity meter 100 has been described in the above embodiments but not limited thereto, and it may be another device or an activity meter such as a pedometer if it can employ the determination result whether or not the walking is an ascending walking or a descending walking on the basis of the acceleration values.

(2) In the above embodiments, whether or not the walking is an ascending walking is determined on the basis of a comparison result between the integrals or pseudo-integrals of the first stance period and the second stance period. However, it is not limited thereto, and it is acceptable to determine whether or not the walking is a descending walking on the basis of the comparison result.

(3) In the above embodiments, the pseudo-integrals are used as the representative values of the first stance period and the second stance period. However, it is not limited thereto, and it may be any identifiable representative value capable of being used to compare the detection values detected by acceleration sensor 170 in the first stance period and the second stance period. For example, it may be an average value over a period of time.

(4) In the first embodiment mentioned above, it is configured that the true or false indicator on the determination result that the walking is an ascending walking is input at two levels, that is, true or false, and the threshold value of the left-right ratio is adjusted in accordance with the input true or false indicator. Specifically, in the case where the determination that the walking is an ascending walking is false, the threshold value of the left-right ratio is increased by 0.01; while in the case where the determination that the walking is a level ground walking is false, the threshold value of the left-right ratio is decreased by 0.01. However, it is not limited thereto, and any other method may be used if it is capable of adjusting the predetermined value for making the determination in accordance with the true or false indicator on the determination result of the walking.

For example, in the case where the walking includes several dozen of level ground walking steps and several dozen of ascending walking steps, the determination result for each of the level ground walking and the ascending walking with the true or false indicators thereof evaluated at 4 levels, that is, “almost true”, “mostly true”, “mostly false” and “almost false” may be input through operation unit 130. Thus, in the level ground walking, in accordance with the input evaluations of “almost true”, “mostly true”, “mostly false” and “almost false”, the threshold value of the left-right ratio may be set as “remaining original”, “remaining original”, “decreasing by 0.01” and “decreasing by 0.02”, respectively. In the ascending walking, in accordance with the input evaluations of “almost true”, “mostly true”, “mostly false” and “almost false”, the threshold value of the left-right ratio may be set as “remaining original”, “remaining original”, “increasing by 0.01” and “increasing by 0.02”, respectively.

(5) In the above embodiments, a three-axis acceleration sensor is used as acceleration sensor 170; however, it is not limited thereto, a uniaxial acceleration sensor or a biaxial acceleration sensor is also applicable to the present invention.

(6) In the above embodiments, the present invention is described as an invention of a physical motion detecting device such as activity meter 100. However, the present invention is not limited thereto, and it may be regarded as an invention of a control method or a control program for controlling the physical motion detecting device.

(7) It should be understood that the embodiments disclosed herein have been presented for the purpose of illustration and description but not limited in all aspects. It is intended that the scope of the present invention is not limited to the description above but defined by the scope of the claims and encompasses all modifications equivalent in meaning and scope to the claims.

### REFERENCE SIGNS LIST

10: user; 100: activity meter; 110: control unit; 120: memory; 130: operation unit; 131: display selection/enter switch; 132: left operation/memory switch; 133: right operation switch; 140: display unit; 141: display; 170: acceleration sensor; 190: power source; 191: main body; 192: clip

1. A physical motion detecting device which comprises a main body equipped with an accelerator sensor for detecting an acceleration of said main body and a control unit, and is configured to detect physical motions of a user with said main body being mounted at a predetermined part of the user,
said control unit including
a detecting member configured to detect whether or not one of said user's feet has touched the ground on the basis of detection values from said acceleration sensor,
in a single walking cycle, a period in which said user is standing on a first foot from a time when said detecting member detects that said first foot has touched the ground to a time when said detecting member detects that a second foot has touched the ground being defined as a first stance period, and
in a single walking cycle, a period in which said user is standing on said second foot from a time when said detecting member detects that said second foot has touched the ground to a time when said detecting member detects that said first foot has touched the ground being defined as a second stance period,
said control unit further including
a calculating member configured to calculate a representative value for the detection values detected by said acceleration sensor for each of said first stance period and said second stance period on the basis of the detection values detected by said acceleration sensor, and
a determining member configured to determine whether or not the walking is an ascending walking or a descending walking on the basis of a comparison result between said representative values calculated by said calculating member for said first stance period and said second stance period.

2. The physical motion detecting device according to claim 1, wherein said determining member determines whether or not the walking is an ascending walking or a descending walking on the basis of a comparison result whether or not a ratio of said representative value for said first stance period relative to said representative value for said second stance period is equal to or greater than a predetermined value.

3. The physical motion detecting device according to claim 2, further comprising
a notification unit configured to notify the user of a piece of predetermined information, and
an input unit configured to receive an input of a piece of predetermined information from the user,
wherein said control unit further includes
a notification controlling member configured to control said notification unit to notify the user of a result determined by said determining member,
an input receiving member configured to receive from said input unit an input of a piece of true or false information representing a true or false indicator on the result notified by said notification member, and
an adjusting member configured to adjust said predetermined value according to the true or false indicator represented by the piece of true or false information received by said input receiving member.

4. The physical motion detecting device according to claim 2, further comprising an input unit configured to receive an input of a piece of predetermined information from the user,
wherein said control unit further includes
an input receiving member configured to receive from said input unit an input for altering said predetermined value, and
an adjusting member configured to alter said predetermined value according to the input received by said input receiving member.

5. The physical motion detecting device according to claim 2, wherein said predetermined value is a value preliminarily determined according to a statistical approach.

6. The physical motion detecting device according to claim 1, wherein said calculating member calculates an integral of the detection values for each of said first stance period and said second stance period as said representative value.

7. The physical motion detecting device according to claim 1, wherein said calculating member calculates a value by using a maximum value and a minimum value of the detection values for each of said first stance period and said second stance period as said representative value.

8. A control method for controlling a physical motion detecting device which comprises a main body equipped with an accelerator sensor for detecting an acceleration of said main body and a control unit, and is configured to detect physical motions of a user with said main body being mounted at a predetermined part thereof,
said control unit being configured to perform
a step of detecting whether or not one of said user's feet has touched the ground on the basis of detection values from said acceleration sensor,
in a single walking cycle, a period in which said user is standing on a first foot from a time when it is detected that said first foot has touched the ground to a time when it is detected that a second foot has touched the ground being defined as a first stance period, and
in a single walking cycle, a period in which said user is standing on said second foot from a time when it is detected that said second foot has touched the ground to a time when it is detected that said first foot has touched the ground being defined as a second stance period,
said control unit being configured to further perform
a step of calculating a representative value for the detection values detected by said acceleration sensor for each of said first stance period and said second stance period on the basis of the detection values detected by said acceleration sensor sensor, and
a step of determining whether the walking is an ascending walking or a descending walking on the basis of a comparison result between said representative values calculated for said first stance period and said second stance period.
